(19) United States
(12) Patent Application Publication
(10) Pub. No.: US 2014/0121499 A1
(43) Pub. Date: May 1, 2014

(54) PATIENT POSITIONING DEVICE FOR PRONE BREAST SIMULATION AND RADIATION THERAPY

(71) Applicant: Qfix Systems, LLC, Avondale, PA (US)

(72) Inventors: Daniel D. Coppens, Avondale, PA (US); David M. Rabeno, Avondale, PA (US); Kristin Gagliardi, Elkton, MD (US); Sean McGrenaghan, Downingtown, PA (US)

(73) Assignee: Qfix Systems, LLC, Avondale, PA (US)

(21) Appl. No.: 14/065,099

(22) Filed: Oct. 28, 2013

Related U.S. Application Data
(60) Provisional application No. 61/719,335, filed on Oct. 26, 2012.

(51) Int. Cl. A61B 5/055; A61N 2/02

(52) U.S. Cl. CPC A61B 5/0555; A61N 2/02
USPC 600/422

(57) ABSTRACT

A patient positioning device for use in simulation and treatment via radiation therapy of the ipsilateral breast of a patient while in the prone position. The patient positioning device has (A) an MRI coil support system comprising (a) at least one of (1) a first MRI coil support structure at the superior end of the device, and (2) a second MRI coil support structure at the inferior end of the device, wherein the first and/or second support structures are dimensioned and configured to receive and engage a first MRI coil at one or more positions and oriented in the vertical plane, and (b) an MRI support assembly comprising a housing dimensioned and configured to receive and engage a second MRI coil, wherein the MRI support assembly is configured to allow adjustment of the position of the housing relative to the one or more positions for receiving and engaging the first MRI coil, (B) an aperture through which the ipsilateral breast can hang into a radiation therapy treatment field, and (C) a provision to support the contralateral breast such that it is held out of the radiation therapy treatment field. Both the first and second MRI coils are positioned such that neither comes in physical contact with the ipsilateral breast.

### CROSS-REFERENCE TO RELATED APPLICATIONS

This application claims the benefit of U.S. Provisional Patent Application Ser. No. 61/719,335, filed Oct. 26, 2012, which is incorporated herein by reference in its entirety.

### TECHNICAL FIELD

The present disclosure generally relates to patient positioning devices for prone breast imaging and treatment and related methods of using such devices in prone breast diagnostic imaging and treatment. More particularly, the present disclosure relates to such patient positioning devices that are configured to allow for both prone breast MRI simulation and radiation therapy while maintaining the ipsilateral breast in substantially the same position for radiation therapy as it was in for simulation.

### BACKGROUND

Radiation therapy is a commonly used approach for treating certain cancers, such as, for example, breast cancer. For example, a cancerous tumor in breast tissue can be destroyed by bombardment with x-ray radiation. Generally, treatment of a tumor by radiation therapy is preceded by a diagnostic imaging procedure known as simulation. During simulation, the patient is oriented and positioned in the manner anticipated for treatment using the same positioning and immobilization devices that will be used in treatment. Typically, the patient is imaged and treated in one of two orientations, supine (i.e., face up) or prone (i.e., face down). Given the spatial constraints, prone orientation can be more advantageous for simulation in a closed MRI.

Some techniques for simulation and radiation therapy require that the patient be positioned and immobilized precisely. This is because certain state of the art cancer radiation therapies are increasingly based on the pinpoint application of high-energy radiation, which can be highly tailored to the shape and position of the cancerous tumor. As the size of the treatment beam decreases, the accurate location of the beam becomes much more critical. For example, if a highly tailored treatment beam is off target (e.g., by a few millimeters), it may miss the tumor. Accurate tumor location and treatment spares the surrounding healthy tissue. Thus, current practice provides that it is important that the patient be simulated in substantially the same position that will be used in treatment in an attempt to ensure accurate identification of tumor location.

Because of these new cancer therapy techniques, it has become increasingly desirable to know the location and shape of the tumor accurately when the patient is positioned for treatment. This can be accomplished by ensuring that the body part of the patient that is imaged during simulation is in as close to the same position for radiation therapy as it was for simulation. However, this can be problematic when MRI is used for simulation. For example, when a breast is imaged in the prone orientation using MRI, it is typically in physical contact with one or more MRI coils. These coils are removed prior to subjecting the breast to any subsequent radiation therapy, but in doing so, the position of the breast can change, such that it is no longer in the same position for radiation therapy as it was for simulation. This change in position increases the risk that the treatment beam might miss the tumor and instead expose surrounding healthy tissue to harmful amounts of radiation.

Thus, an interest exists for improved devices for use in both prone breast MRI simulation and radiation therapy such that they avoid a shift in the position of the breast as result of the removal of MRI coils prior to treatment.

### EMBODIMENTS OF THE INVENTION

One embodiment of the present invention is a patient positioning device for use in simulation and treatment via radiation therapy of the ipsilateral breast of a patient while in the prone position comprising
(A) an MRI coil support system comprising
   (a) at least one of:
      (1) a first MRI coil support structure at the superior end of the device; and
      (2) a second MRI coil support structure at the inferior end of the device;
      wherein
      the first and/or second support structures are dimensioned and configured to receive and engage a first MRI coil at one or more positions and oriented in the vertical plane, and
      the first MRI coil is positioned such that it does not come in physical contact with the ipsilateral breast; and
   (b) an MRI support assembly comprising a housing dimensioned and configured to receive and engage a second MRI coil;
      wherein
      the MRI support assembly is configured to allow adjustment of the position of the housing relative to the one or more positions for receiving and engaging the first MRI coil, and
      the second MRI coil is positioned such that it does not come in physical contact with the ipsilateral breast;
(B) an aperture through which the ipsilateral breast can hang into a radiation therapy treatment field; and
(C) a provision to support the contralateral breast such that it is held out of the radiation therapy treatment field.

Another embodiment of the present invention is the above patient positioning device, wherein the MRI support assembly comprises a rail or track and the housing is directly movably engaged to the rail or track.

Another embodiment of the present invention is the above patient positioning device, wherein the MRI support assembly comprises a rail or track and the housing is removably coupled to an attachment member that is movably engaged to the rail or track.

Another embodiment of the present invention is the above patient positioning device, further comprising a base on the anterior side of the MRI coil support system and coupled to the first and/or second MRI coil support structures, wherein the base is positioned such that the breast may contact the base during simulation and treatment.

Another embodiment of the present invention is the above patient positioning device, wherein the base is removably coupled to the first and/or second MRI coil support structures.

Another embodiment of the present invention is the above patient positioning device, wherein the base is dimensioned and configured to allow for insertion and removal of the first MRI coil via the anterior side of the MRI coil support system.

Another embodiment of the present invention is the above patient positioning device, further comprising one or more compartments adjacent to the first and/or second MRI coil support structures.

Another embodiment of the present invention is the above patient positioning device, wherein the first and/or second MRI coil support structures are dimensioned and configured to allow for insertion and removal of the first MRI coil by sliding laterally along the top edges of the first and second MRI coil support structures.

Another embodiment of the present invention is the above patient positioning device, further comprising the first and second MRI coils.

Another embodiment of the present invention is the above patient positioning device, wherein only the first MRI coil support structure is dimensioned and configured to receive and engage the first MRI coil.

Another embodiment of the present invention is the above patient positioning device, wherein only the second MRI coil support structure is dimensioned and configured to receive and engage the first MRI coil.

Another embodiment of the present invention is the above patient positioning device, wherein both the first and the second MRI coil support structures are dimensioned and configured to receive and engage the first MRI coil.

Another embodiment of the present invention is the above patient positioning device, wherein the device further comprises:
(1) a superior patient platform; and
(2) an inferior patient platform;
   wherein
   the superior body is removably coupled to the superior end of the MRI coil support system,
   the inferior body is removably coupled to the inferior end of the MRI coil support system,
   the provision to support the contralateral breast is removably coupled to the posterior side of the MRI coil support system, and
   the provision to support the contralateral breast is dimensioned and configured to define the opening (c).

Another embodiment of the present invention is the above patient positioning device, wherein the provision to support the contralateral breast can be positioned to allow either the right or left breast of a patient to hang through the opening (c).

Another embodiment of the present invention is the above patient positioning device, wherein the posterior surface of the device is substantially flat.

Another embodiment of the present invention is the above patient positioning device, wherein the device comprises indexing positions for a headrest and handholds, wherein the indexing positions are dimensioned and configured so that a headrest and a handhold can be repeatedly positioned in substantially the same locations.

Another embodiment of the present invention is the above patient positioning device, further comprising a headrest and a handhold.

Another embodiment of the present invention is the above patient positioning device, wherein the first and second MRI coils are positioned relative to each other such that, when a patient lies prone on the device and the ipsilateral breast hangs through the opening (c) and between the first and second MRI coils, neither MRI coil comes in contact with the ipsilateral breast

Another embodiment of the present invention is the above patient positioning device, wherein the device is mounted on a couch top.

Another embodiment of the present invention is the above patient positioning device, wherein the device is mounted on a patient transfer assembly.

Another embodiment of the present invention is the above patient positioning device, wherein the patient transfer assembly is releasably mounted on a trolley assembly.

Another embodiment of the present invention is the above patient positioning device, further comprising at least one indexing plate dimensioned and configured such that the device may be repeatedly positioned relative to a couch top.

Another embodiment of the present invention is the above patient positioning device, further comprising at least one indexing feature such that the patient may be repeatedly positioned with respect to the device.

Another embodiment of the present invention is the above patient positioning device, wherein the device is dimensioned and configured so that it can be used in conjunction with one or more simulation techniques and one or more radiotherapy treatment techniques.

Another embodiment of the present invention is the above patient positioning device, wherein the one or more simulation techniques is selected from the group consisting of MRI, CT, and PET.

Another embodiment of the present invention is the above patient positioning device, wherein the one or more radiotherapy treatment techniques is selected from the group consisting of X-ray, photon, and proton.

Another embodiment of the present invention is the above patient positioning device, wherein

the first and/or second support structures are dimensioned and configured to receive and engage a first MRI coil at one or more positions adjacent and in an orientation at least substantially parallel to the axis of the patient; and
and the housing is in an orientation at least substantially parallel to the one or more positions for receiving and engaging the first MRI coil.

Another embodiment of the present invention is the above patient positioning device, wherein the first and/or second support structures are dimensioned and configured to receive and engage an MRI coil at one or more positions in an orientation at least substantially perpendicular to the axis of the patient.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features and combinations of features described below and illustrated in the figures can be arranged and organized differently to result in embodiments which are still within the spirit and scope of the present disclosure. To assist those of ordinary skill in the art in making and using the disclosed systems, assemblies and methods, reference is made to the appended figures, wherein:

FIG. 1 is a partial top-side perspective view of an exemplary patient positioning device according to the present invention, wherein the provision to support the contralateral breast has been omitted.

FIG. 2 is a partial top-side perspective view of an exemplary patient positioning device according to the present invention, wherein the MRI support system has been omitted.

FIG. 3 is a top-side perspective view of an exemplary patient positioning device according to the present invention.

FIG. 4 is a top-side perspective view of an exemplary patient positioning device according to the present invention, wherein a patient is depicted lying prone on the device.

FIG. 5 is a bottom-side perspective view of an exemplary patient positioning device according to the present invention.

FIG. 6 is a partial bottom-side perspective view of an exemplary patient positioning device according to the present invention.

FIG. 7 is a partial top-side perspective view of an exemplary MRI support system for use in the patient positioning devices according to the present invention, wherein the housing of the MRI support assembly has been omitted.

FIG. 8 is a partial top-side perspective view of an exemplary MRI support system for use in the patient positioning devices according to the present invention, wherein the housing of the MRI support assembly has been omitted.

FIG. 9 is a top-side perspective view of an exemplary MRI support system for use in the patient positioning devices according to the present invention.

FIG. 10 is a bottom-side perspective view of an exemplary MRI support system for use in the patient positioning devices according to the present invention.

FIG. 11 is a side perspective view of an exemplary patient positioning device according to the present invention being used in conjunction with an MRI machine.

FIG. 12 is a side perspective view of an exemplary patient positioning device according to the present invention being used in conjunction with a radiation therapy machine.

### DETAILED DESCRIPTION OF THE INVENTION

The exemplary embodiments disclosed herein are illustrative of the improved patient positioning devices of the present invention. However, it should be understood that the disclosed embodiments are merely exemplary of the present invention, which may be embodied in various forms. Therefore, details disclosed herein with reference to exemplary patient positioning devices and associated processes or techniques of their assembly and use are not to be interpreted as limiting, but merely as the basis for teaching one skilled in the art how to make and use the patient positioning devices of the present invention.

The present disclosure provides for improved patient positioning devices. More particularly, the present disclosure provides for improved patient positioning devices that are configured for both prone breast MRI simulation and radiation therapy while maintaining the ipsilateral breast in substantially the same position for radiation therapy as it was in for simulation. The patient positioning device of the present invention for use in simulation and treatment via radiation therapy of the ipsilateral breast of a patient while in the prone position comprises:
(A) an MRI coil support system comprising
   (a) at least one of:
      (1) a first MRI coil support structure at the superior end of the device; and
      (2) a second MRI coil support structure at the inferior end of the device;
      wherein
      the first and/or second support structures are dimensioned and configured to receive and engage a first MRI coil at one or more positions and oriented in the vertical plane, and
   (b) an MRI support assembly comprising a housing dimensioned and configured to receive and engage a second MRI coil;
      wherein
      the MRI support assembly is configured to allow adjustment of the position of the housing relative to the one or more positions for receiving and engaging the first MRI coil, and
(B) an aperture through which the ipsilateral breast can hang into a radiation therapy treatment field; and
(C) a provision to support the contralateral breast such that it is held out of the radiation therapy treatment field.

Both the first and second MRI coils are positioned such that they do not come in physical contact with the ipsilateral breast subject to simulation and treatment. This configuration allows for the removal of MRI coils from the MRI coil support system without disturbing the position of the breast, thereby avoiding a shift in the position of the breast prior to radiation therapy.

Referring now to the drawings, like parts are marked throughout the specification and drawings with the same reference numerals, respectively. Drawing figures are not necessarily to scale and in certain views, parts may have been exaggerated for purposes of clarity.

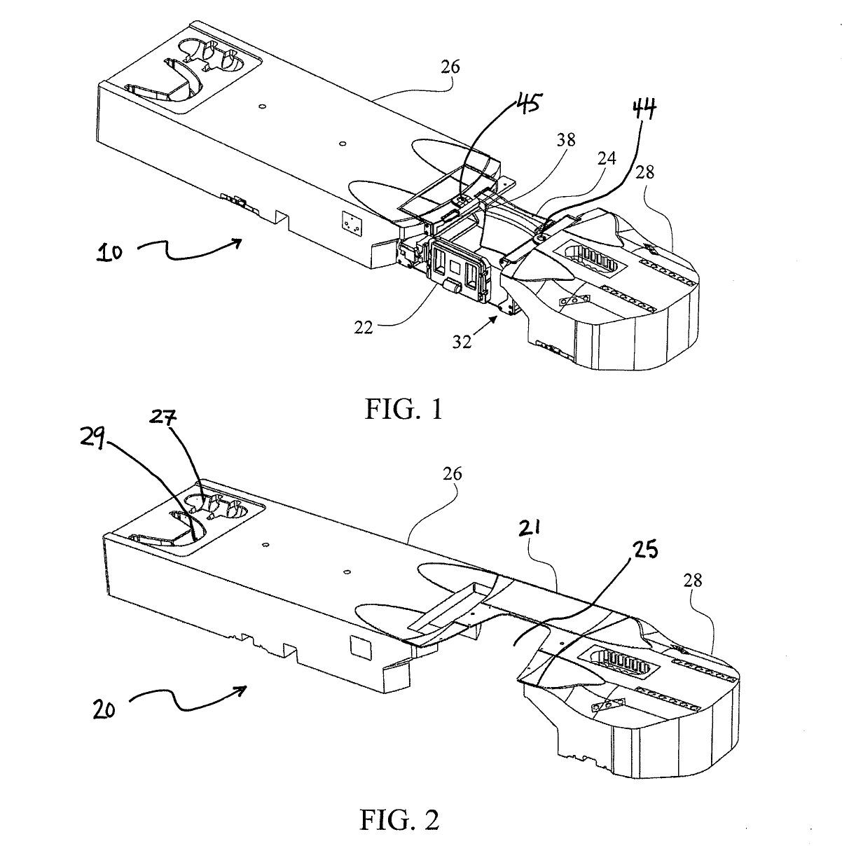

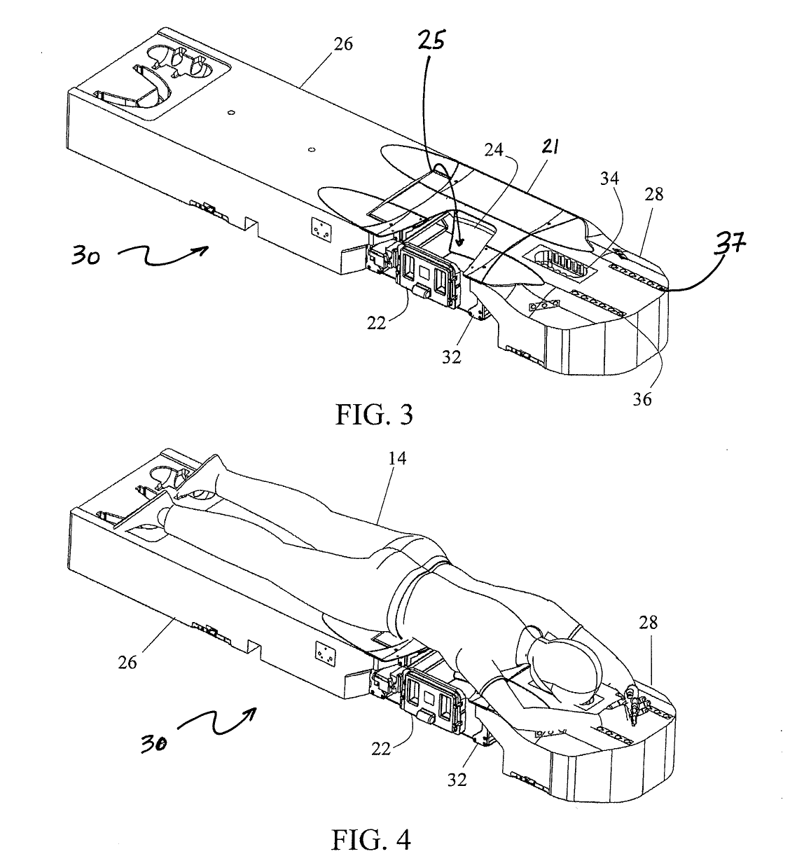

With reference to FIGS. 1 and 2, there are illustrated partial exemplary embodiments of the patient positioning device according to the present invention. The provision to support the contralateral breast 21 has been omitted from the patient positioning device 10 in FIG. 1, while the MRI support system 32 has been omitted from the patient positioning device 20 in FIG. 2. With reference to FIGS. 3 and 4, there are illustrated exemplary embodiments of the patient positioning device 30 according to the present invention. A patient 14 is depicted lying prone on the device 30 in FIG. 4.

In FIG. 3, the patient positioning device 30 comprises a superior patient platform 28, an inferior patient platform 26, an MRI support system 32, and a provision to support the contralateral breast 21. The patient positioning device 30 is assembled by (1) removably coupling the inferior end of superior patient platform 28 to the superior end of MRI support system 32, (2) removably coupling the inferior end of MRI support system 32 to the superior end of inferior patient platform 26, and (3) removably coupling the provision to support the contralateral breast 21 to the posterior side of the MRI support system 32. In other embodiments, the patient positioning devices of the present invention can comprise alternative patient platforms to those of FIGS. 2-4. For example, the patient platform can be a single platform having a continuous surface that includes a provision to support the contralateral breast. In a further embodiment, the provision to support the contralateral breast of the single patient platform having a continuous surface can be removable and reversible.

In the case of patient positioning device 30, removable coupling is achieved by sliding a portion of the inferior end of the superior patient platform 28 over a portion of the superior end of the MRI support system 32, sliding a portion of the superior end of the inferior patient platform 26 over a portion of the inferior end of MRI support system 32, and attaching the provision to support the contralateral breast 21 to the MRI support system 32 at two attachment points 44 and 45, shown in FIG. 1, although the present invention is not limited thereto. The provision to support the contralateral breast 21 is dimensioned and configured to have an aperture 25 through which the ipsilateral breast of a patient lying in the prone orientation can hang into a radiation therapy treatment field and between the first and second MRI coils 24 and 22.

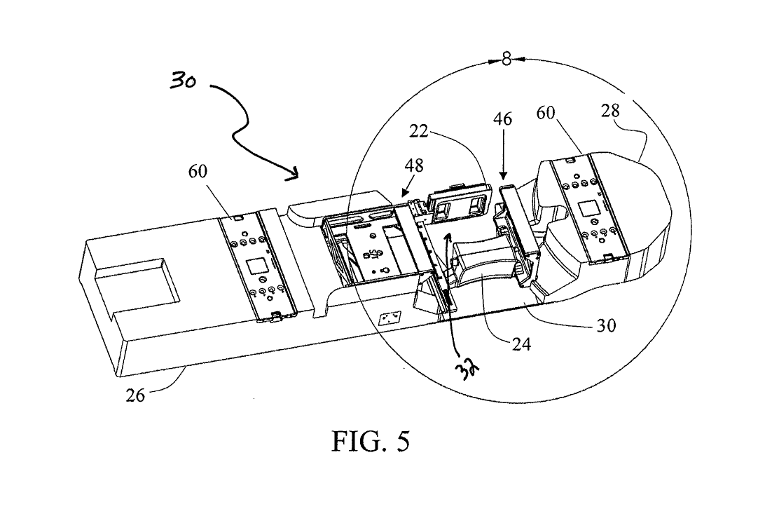

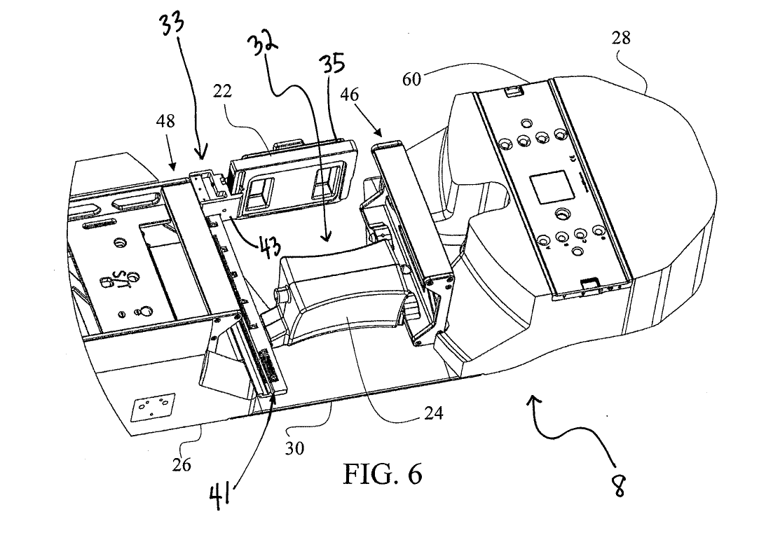

With reference to FIG. 5, there is illustrated a view from the anterior side of an exemplary embodiment of the patient positioning device according to the present invention. With reference to FIG. 6, there is illustrated an enlarged view from the anterior side 8 of the exemplary patient positioning device according to the present invention as shown in FIG. 5.

As shown in FIGS. 5 and 6, the patient positioning device 30 comprises an MRI coil support system 32, which comprises both a first MRI support structure 46 at the superior end of the device and a second MRI support structure 48 at the inferior end of the device. However, the patient positioning devices of the present invention are not required to have both support structures and, in alternative embodiments, can instead comprise an MRI support structure at the superior end of the device only or an MRI support structure at the inferior end of the device only. The first and second support structures 46 and 48 are dimensioned and configured to receive and engage a first MRI coil 24 at a position adjacent and at least substantially parallel to the axis of a patient lying prone on the patient positioning device 30. As shown in FIG. 6, the MRI coil support system 32 comprises an MRI support assembly 33 comprising a housing 35 dimensioned and configured to receive and engage a second MRI coil 22. The MRI support assembly 33 is configured to allow adjustment of the position of the housing 35, and thus the second MRI coil 22, relative to the positions on the first and second support structures 46 and 48 for receiving and engaging the first MRI coil 24 along a track 41, to which the housing 35 is movably engaged via an attachment member 43. In this way, the first and second MRI coils 24 and 22 can be positioned relative to each other such that neither comes in contact with the ipsilateral breast of the patient. In the MRI coil support system 32, the housing 35 is removably coupled to the attachment member 43. However, in an alternative embodiment, the housing 35 could be directly movably engaged to the track 41.

In one alternative embodiment, the first and/or second support structures are dimensioned and configured to receive and engage a first MRI coil at one or more positions adjacent and in an orientation at least substantially parallel to the axis of the patient and the housing is in an orientation at least substantially parallel to the one or more positions for receiving and engaging the first MRI coil. In another alternative embodiment, the first and/or second support structures are dimensioned and configured to receive and engage an MRI coil at one or more positions in an orientation at least substantially perpendicular to the axis of the patient.

The provision to support the contralateral breast 21 of patient positioning device 30, is removable and reversible, although the present invention is not limited thereto and non-removable, non-reversible provisions to support the contralateral breast are also contemplated by the present invention. Thus, when the provision to support the contralateral breast 21 is positioned as shown in FIGS. 2 and 3 and a patient is lying prone on the patient positioning device 30, the left breast of the patient would rest on the surface of the provision to support the contralateral breast 21 (i.e., the contralateral breast), while the right breast of the patient would hang through the opening 25 created by the dimensions of the provision to support the contralateral breast 21 (i.e., the ipsilateral breast). When the position of the reversible provision to support the contralateral breast 21 is reversed, the right breast of the patient is then the contralateral breast, while the left breast of the patient is then the ipsilateral breast.

As shown in FIG. 3, when the superior patient platform 28, inferior patient platform 26, MRI support system 32, and provision to support the contralateral breast 21 of this exemplary embodiment is removably coupled, the posterior surface of patient positioning device 30 is substantially flat, although the present invention is not limited thereto. In alternative embodiments, the patient platforms and the provisions to support the contralateral breast of the patient positioning devices of the present invention can be dimensioned and configured such that the posterior surface of the apparatus takes on various shapes and contours. For example, the provision to support the contralateral breast of the patient can be ergonomically contoured to provide a more comfortable position for the patient and to reduce pressure on the contralateral breast.

As shown in FIG. 3, the superior patient platform 28 of the patient positioning device 30 comprises a headrest indexing position 34 for mounting a headrest as well as one or more handhold indexing positions 36 and 37 for mounting handholds, although the present invention is not limited thereto. The headrest and handhold indexing positions are dimensioned and configured so that a headrest and handholds can be repeatably positioned in substantially the same location on the patient positioning device 30 for given patient being subjected to simulation and radiotherapy treatment. Thus, the patient positioning devices of the present invention may also comprise a headrest and handholds attached to the patient platform. The patient positioning devices of the present invention can also comprise storage compartments for storing the headrest and handholds, such as storage compartments 27 and 29 located at the end of inferior patient platform 26, as shown in FIG. 2.

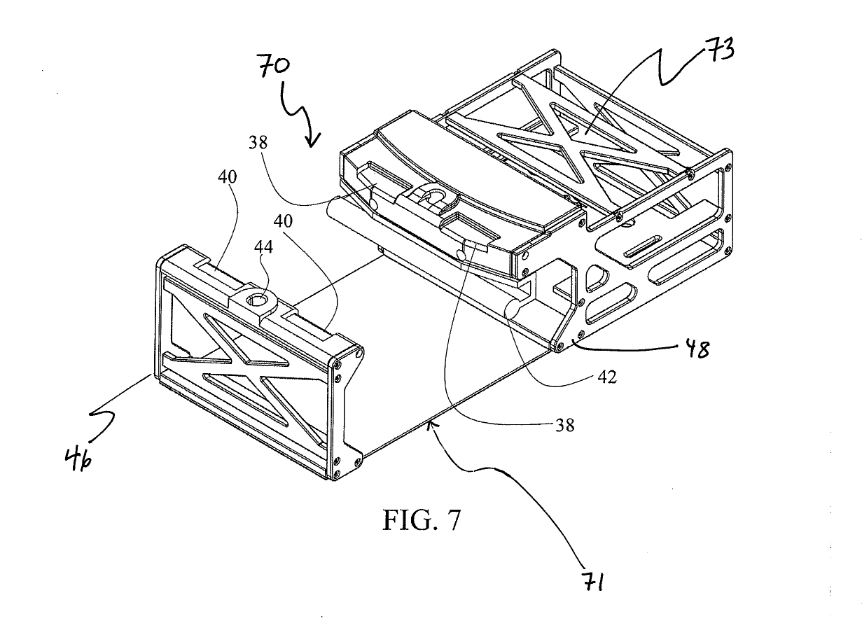

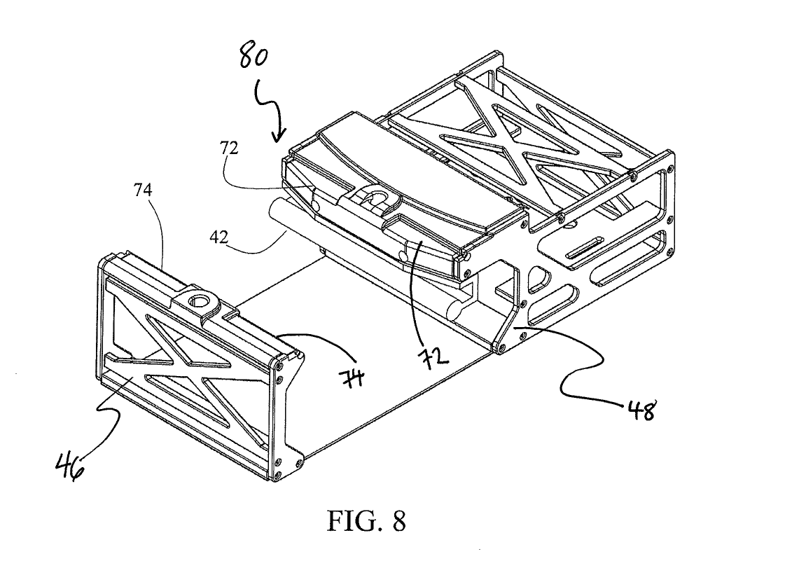

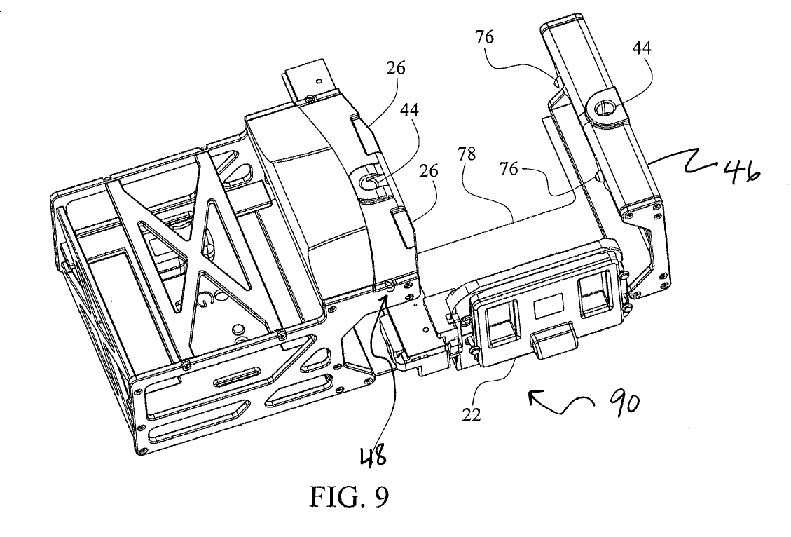

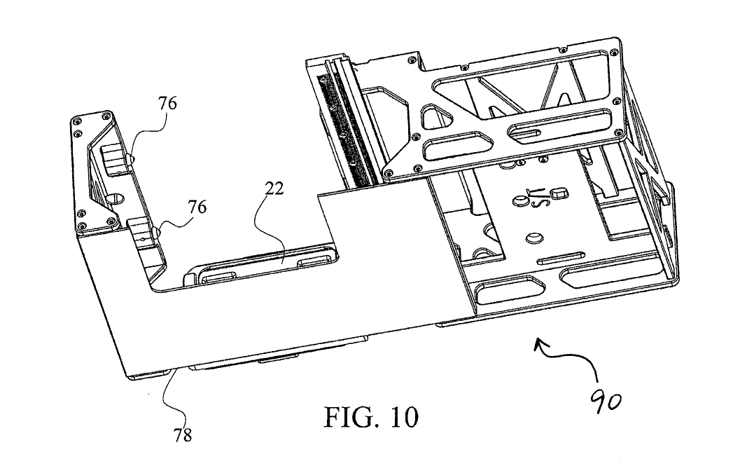

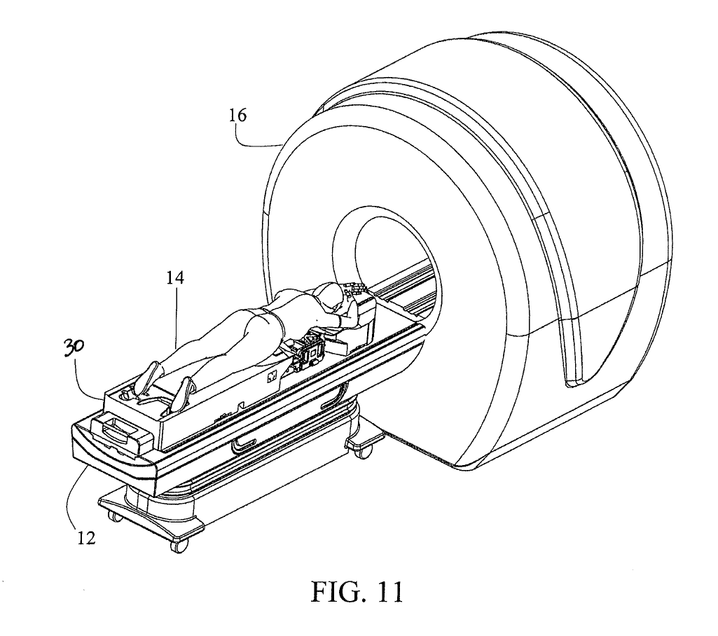

With reference to FIGS. 7 and 8, there are illustrated partial exemplary embodiments of an MRI support system for use in the patient positioning devices according to the present invention. The housing of the MRI support assembly has been omitted from FIGS. 7 and 8. With reference to FIGS. 9 and 10, there is illustrated top and bottom views of an exemplary MRI support system for use in the patient positioning devices according to the present invention.

The exemplary MRI support system 70 of FIG. 7 comprises a first support structure 46 at the superior end of the MRI support system 70 and a second support structure 48 at the inferior end of the MRI support system 70. Both first and second support structures 46 and 48 comprise notches 38 and 40, which are dimensioned and configured to receive and engage an MRI coil. While both the first and second support structures 46 and 48 of MRI support system 70 are dimensioned and configured to receive and engage an MRI coil 24, it is within the scope of this invention that only the first support structure 46 or the second support structure 48 are so dimensioned and configured, such that the first MRI coil 24 only engages the first support structure 46 or the second support structure 48. In an alternative embodiment, each of the first and/or second support structures 46 and 48 can comprise only one such notch or three or more such notches.

The exemplary MRI support system 70 of FIG. 7 also comprises a base 17 on its anterior side, although MRI support systems for use in the patient positioning devices according to the present invention with no base on its anterior side are also contemplated as part of the present invention. The base 71 is coupled to the first and second support structures 46 and 48 and, as alternative embodiments, can either be permanently affixed or removably coupled to one or both support structures. In a further embodiment, the base can be positioned on the anterior side of the MRI support system such that it comes in contact with the ipsilateral breast of the patient.

The exemplary MRI support system 70 of FIG. 7 also comprises a compartment 73 adjacent to the second support structure, although MRI support systems for use in the patient positioning devices according to the present invention with no such compartment are also contemplated as part of the present invention. In an alternative embodiment, such a compartment can instead be adjacent to the first support structure. These compartments can be used to house electronic equipment used in conjunction with the MRI coils, such as a CPU. Therefore, such compartments can also comprise openings through which cables from the electronic equipment to the MRI coils can pass.

In an alternative embodiment, the first and second support structures 46 and 48 of MRI support system 80 of FIG. 8 can comprise edge surfaces 72 and 74. These edge surfaces allow for insertion and removal of the first MRI coil 24 by laterally sliding the first MRI coil 24 along the edge surfaces 72 and 74. In a further embodiment, the support structures and/or the first MRI coil 24 can be equipped with a mechanism that removably locks the first MRI coil 24 into place on the MRI support system 80.

As shown in FIGS. 8 and 9, the base, whether removable or fixed, can be dimensioned and configured to allow for insertion and removal of the first MRI coil via the anterior side of the MRI support system. In these Figures, the base 78 of MRI support system 90 has a “windowed” configuration, i.e., there is an opening on one side of the base. This “windowed” configuration facilitates access for insertion and removal of the first MRI coil 24 via the anterior side of the MRI support system 90, while at the same time providing a contact surface for the ipsilateral breast of the patient. In MRI support system 90, the first MRI coil 24 is to be held in place, in part, on the first support structure 46 by one of retractable pins 76. Thus, the first MRI coil 24 is removed by pulling the pinned side of the coil in the anterior direction and out and off of the notch 26 on the second support structure 48. Insertion of the first MRI coil 24 is achieved by performing these steps in reverse, i.e., the first MM coil 24 is inserted into notch 26 and then pressed into the retractable pin 76 until the pin engages the first MRI coil 24. Alternatively, such retractable pins 76 can be located on either the second support structure 48 or on both the first and second support structures 46 and 48. In an alternative embodiment, base 78 is reversible, so that it can be positioned, depending on whether the right or left breast of the patient is the ipsilateral breast, with the “window” on the right or left of the anterior side of the MRI support system.

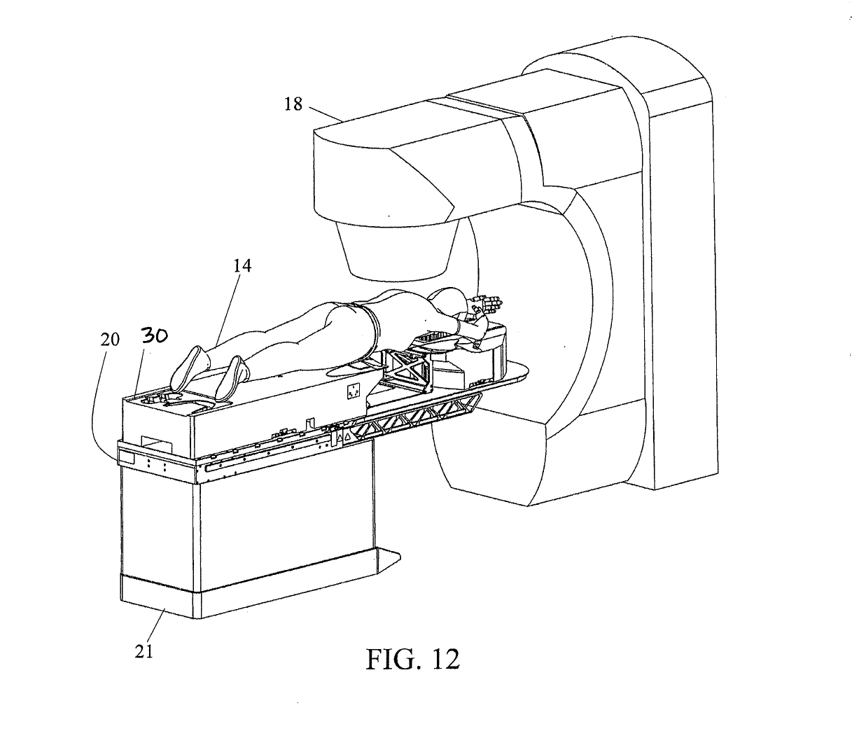

With reference to FIG. 10, there is illustrated an exemplary embodiment of the patient positioning device 30 of the present invention being used in conjunction with an MRI machine 16. The patient positioning device 30 is mounted to an MRI table 12 and a patient 14 is lying prone on the patient positioning device 30. With reference to FIG. 11, there is illustrated an exemplary embodiment of the patient positioning device 30 of the present invention being used in conjunction with a linear accelerator 18. The patient positioning device 30 is mounted to a treatment couch top 20, which is in turn mounted to a treatment couch 21. A patient 14 is lying prone on the patient positioning device 30 to receive radiotherapy treatment. It should be noted from FIG. 12 that the MRI coils have been removed from the MRI support system.

As shown in FIGS. 9 and 10, the patient positioning devices of the present invention are configured and dimensioned such that can be used in conjunction with one or more simulation techniques and one or more radiotherapy treatment techniques. Such simulation techniques include MRI, CT, and PET. Such radiotherapy treatment techniques include X-ray, photon, and proton. The apparatus can be mounted on a couch top, table top, or on any patient transfer assembly. The patient transfer assembly can be releasably mounted to on a trolley assembly that can be used to transport the patient from simulation to radiotherapy treatment without having the prone patient change position (i.e., by getting up from the positioning apparatus at some time point between simulation to radiotherapy treatment).

As shown in FIGS. 5 and 6, the patient positioning device of the present invention can comprise one or more indexing plates 60 dimensioned and configured such that the device may be repeatedly positioned relative to a couch top, table top, or on any patient transfer assembly. The patient positioning device of the present invention can also further comprise at least one indexing feature such that the patient may be repeatedly positioned with respect to the device.

Another embodiment of the present invention is the modification, in accordance with the present invention, of cantilevered prone patient positioning devices, such as those disclosed in U.S. Patent App. Pub. No. 2011/0200178 A1, which is incorporated herein by reference in its entirety. In one embodiment, the MRI support system used in the patient positioning devices according to the present invention is removably coupled to the bottom of the cantilevered device. The first and/or second support structures of the MRI support system are directly, removably coupled to positions on the anterior side of the cantilevered device that are superiorly and/or inferiorly located relative to both the cantilever portion of the device and the aperture created by the shape of the cantilever portion (i.e., the beast cutout). Alternatively, in another embodiment, the anterior side of cantilever portion of the device is dimensioned and configured to receive and engage a first MRI coil in such a manner that it is adjacent and substantially parallel to the axis of a patient lying prone on the device, while an MRI support assembly comprising a housing dimensioned and configured to receive and engage a second MRI coil and to allow adjustment of the position of the housing relative to the position of the first MRI coil is removably coupled to the device at a position superiorly or inferiorly located relative to the aperture created by the dimensions of the cantilever portion of the device. In both embodiments, both the first and second MRI coils can be positioned relative to each other such that when a patient lies prone on the device, the contralateral breast lies on the cantilever portion of the device, while the ipsilateral breast hangs through the opening and between the first and second MRI coils. The first and second MRI coils are positioned to the left and right of the ipsilateral breast, but neither come in contact with the ipsilateral breast.

Whereas the disclosure has been described principally in connection with improved patient positioning devices for hospital, treatment center, and/or commercial uses/purposes, such description has been utilized only for purposes of disclosure and is not intended as limiting the disclosure. To the contrary, it is to be recognized that the disclosed patient positioning devices are capable of use in other applications and for other purposes.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A patient positioning device for use in simulation and treatment via radiation therapy of the ipsilateral breast of a patient while in the prone position comprising
(A) an MRI coil support system comprising
(a) at least one of:
(1) a first MRI coil support structure at the superior end of the device; and
(2) a second MRI coil support structure at the inferior end of the device;
wherein
the first and/or second support structures are dimensioned and configured to receive and engage a first MRI coil at one or more positions and oriented in the vertical plane, and
the first MRI coil is positioned such that it does not come in physical contact with the ipsilateral breast; and
(b) an MRI support assembly comprising a housing dimensioned and configured to receive and engage a second MRI coil;
wherein
the MRI support assembly is configured to allow adjustment of the position of the housing relative to the one or more positions for receiving and engaging the first MRI coil,
the second MRI coil is positioned such that it does not come in physical contact with the ipsilateral breast;
(B) an aperture through which the ipsilateral breast can hang into a radiation therapy treatment field; and
(C) a provision to support the contralateral breast such that it is held out of the radiation therapy treatment field.

2. The patient positioning device of claim 1, wherein the MRI support assembly comprises a rail or track and the housing is directly movably engaged to the rail or track.

3. The patient positioning device of claim 1, wherein the MRI support assembly comprises a rail or track and the housing is removably coupled to an attachment member that is movably engaged to the rail or track.

4. The patient positioning device of claim 1, further comprising a base on the anterior side of the MRI coil support system and coupled to the first and/or second MRI coil support structures, wherein the base is positioned such that the breast may contact the base during simulation and treatment.

5. The patient positioning device of claim 4, wherein the base is removably coupled to the first and/or second MRI coil support structures.

6. The patient positioning device of claim 4, wherein the base is dimensioned and configured to allow for insertion and removal of the first MRI coil via the anterior side of the MRI coil support system.

7. The patient positioning device of claim 1, further comprising the first and second MRI coils.

8. The patient positioning device of claim 1, wherein only the first MRI coil support structure is dimensioned and configured to receive and engage the first MRI coil.

9. The patient positioning device of claim 1, wherein only the second MRI coil support structure is dimensioned and configured to receive and engage the first MRI coil.

10. The patient positioning device of claim 1, wherein both the first and the second MRI coil support structures are dimensioned and configured to receive and engage the first MRI coil.

11. The patient positioning device of claim 1, wherein the device further comprises:
(1) a superior patient platform; and
(2) an inferior patient platform;
wherein
the superior patient platform is removably coupled to the superior end of the MRI coil support system,
the inferior patient platform is removably coupled to the inferior end of the MRI coil support system,
the provision to support the contralateral breast is removably coupled to the posterior side of the MRI coil support system, and
the provision to support the contralateral breast is dimensioned and configured to define the opening (c).

12. The patient positioning device of claim 1, wherein the provision to support the contralateral breast can be positioned to allow either the right or left breast of a patient to hang through the opening (c).

13. The patient positioning device of claim 1, wherein the device comprises indexing positions for a headrest and handholds, wherein the indexing positions are dimensioned and configured so that a headrest and a handhold can be repeatedly positioned in substantially the same locations.

14. The patient positioning device of claim 7, wherein the first and second MRI coils are positioned relative to each other such that, when a patient lies prone on the device and the ipsilateral breast hangs through the opening (c) and between the first and second MRI coils, neither MRI coil comes in contact with the ipsilateral breast

15. The patient positioning device of claim 1, wherein the device is mounted on a couch top.

16. The patient positioning device of claim 1, further comprising at least one indexing plate dimensioned and configured such that the device may be repeatedly positioned relative to a couch top.

17. The patient positioning device of claim 1, further comprising at least one indexing feature such that the patient may be repeatedly positioned with respect to the device.

18. The patient positioning device of claim 1, wherein the device is dimensioned and configured so that it can be used in conjunction with one or more simulation techniques and one or more radiotherapy treatment techniques.

19. The patient positioning device of claim 1, wherein
the first and/or second support structures are dimensioned and configured to receive and engage a first MRI coil at one or more positions adjacent and in an orientation at least substantially parallel to the axis of the patient; and
and the housing is in an orientation at least substantially parallel to the one or more positions for receiving and engaging the first MRI coil.

20. The patient positioning device of claim 1, wherein the first and/or second support structures are dimensioned and configured to receive and engage an MRI coil at one or more positions in an orientation at least substantially perpendicular to the axis of the patient.
